(19) United States
(12) Patent Application Publication
(10) Pub. No.: US 2021/0330180 A1
(43) Pub. Date: Oct. 28, 2021

(54) CAPSULE ENDOSCOPE SYSTEM AND RECEIVING DEVICE

(71) Applicant: OLYMPUS CORPORATION, Tokyo (JP)

(72) Inventors: Naoto KOIDE, Tokyo (JP); Shinichi NAKAJIMA, Tokyo (JP)

(73) Assignee: OLYMPUS CORPORATION, Tokyo (JP)

(21) Appl. No.: 17/371,444

(22) Filed: Jul. 9, 2021

Related U.S. Application Data
(63) Continuation of application No. PCT/JP2019/000744, filed on Jan. 11, 2019.

(51) Int. Cl. A61B 1/04; A61B 1/00

(52) U.S. Cl. CPC A61B 1/041; A61B 1/00011

(57) ABSTRACT

A receiving device transmits and receives a radio signal by radio communication with a capsule endoscope introduced inside a subject. The receiving device includes: a first receiver configured to selectively receive the radio signal by switching between a first frequency and a second frequency; a second receiver configured to receive positioning information from an external positioning system; a processor configured to acquire information indicating whether the positioning information has been received from the positioning system, and a signal level of a signal at, at least one of the first frequency and the second frequency, and set to either one of a mode in which the first frequency is used for the radio communication and a mode in which the second frequency is used for the radio communication; and a first transmitter configured to transmit information of frequency according to the set mode to the capsule endoscope.

This application is a continuation of International Application No. PCT/JP2019/000744, filed on Jan. 11, 2019, the entire contents of which are incorporated herein by reference.

### BACKGROUND

The present disclosure relates to a capsule endoscope system and a receiving device.

Endoscopes have been widely diffused as a medical observation device that is introduced into a body of a subject, such as a patient, to observe the inside of the body of the subject. Moreover, in recent years, a capsule endoscope that is a swallowed image acquiring device including, inside a capsule casing, an imaging device and a communication device that wirelessly transmits an image signal captured by the image acquiring device to an outside of the body, and the like has been developed. The capsule endoscope moves inside organs, such as, an esophagus, a stomach, and a small intestine, with peristaltic movement after it is swallowed from a mouth of a subject for observation inside the body of the subject until it is naturally discharged out from the subject, and sequentially performs imaging.

Image data captured by the capsule endoscope while moving inside the body of the subject is sequentially transmitted to an outside of the body by radio communication, and is stored in a memory provided inside or outside a receiving device arranged outside the body, or is displayed on a display provided in the receiving device. A doctor or a nurse may take the image data stored in the memory into an image processing apparatus through a cradle in which the receiving device is inserted, and may diagnose based on an image displayed on a display of the image processing apparatus.

If a radio interference occurs in communication, a noise may be superimposed on the image data, and it may cause a missing part in the image. Therefore, it is desirable to avoid a radio interference in communication. To cope with this demand, a technique in which a position detection is performed by the global positioning system (GPs) and a radio frequency to be used for radio communication is set depending on the area has been known (for example, refer to JP-A 2005-287685). Moreover, a communication method in which when a radio frequency to be used overlaps with that of other devices, setting the radio frequency to be used to a frequency not overlapping therewith has been known (for example, refer to JP-A-2014-22999). By applying these techniques disclosed in JP-A 2005-287685 and JP-A-2014-22999 in combination to the capsule endoscope and the receiving device, interference caused by a radio interference may be suppressed.

### SUMMARY

According to one aspect of the present disclosure, there is provided a receiving device for transmitting and receiving a radio signal by radio communication with a capsule endoscope introduced inside a subject, the receiving device including: a first receiver configured to selectively receive the radio signal by switching between a first frequency and a second frequency that is different from the first frequency; a second receiver configured to receive positioning information from an external positioning system; a processor including hardware, the processor being configured to acquire information indicating whether the positioning information has been received from the positioning system, and a signal level of at least one of the first frequency and the second frequency, and set to either one of a mode in which the first frequency is used for the radio communication and a mode in which the second frequency is used for the radio communication; and a first transmitter configured to transmit information of frequency according to the set mode to the capsule endoscope.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a schematic diagram illustrating a schematic configuration of a capsule endoscope system according to an embodiment;

FIG. 2 is a block diagram illustrating a schematic configuration of the capsule endoscope system according to the embodiment;

FIG. 3 is a block diagram illustrating a configuration of an essential part of a capsule endoscope in the capsule endoscope system according to the embodiment;

FIG. 4 is a block diagram illustrating a configuration of an essential part of a receiving device in the capsule endoscope system according to the embodiment;

FIG. 5 is a flowchart of mode setting processing performed by the capsule endoscope system according to the embodiment;

FIG. 6 is a flowchart of processing of Mode 1 in the mode setting processing performed by the capsule endoscope system according to the embodiment;

FIG. 7 is a flowchart of Mode 2 in the mode setting processing performed by the capsule endoscope system according to the embodiment;

FIG. 8 is a flowchart of mode 3 in the mode setting processing performed by the capsule endoscope system according to the embodiment;

FIG. 9 is a flowchart of mode 3A in the mode setting processing performed by the capsule endoscope system according to the embodiment;

FIG. 10 is a flowchart of mode 4 in the mode setting processing performed by the capsule endoscope system according to the embodiment; and

FIG. 11 is a diagram for explaining a mode that may be set in an external environment.

### DETAILED DESCRIPTION

Hereinafter, a capsule endoscope system that uses a medical capsule endoscope will be explained as an embodiment. Like reference signs are assigned to like parts throughout the drawings. Moreover, the drawings are schematic illustrations, and it is noted that a relation between a thickness and a width of respective members, ratios of the respective members differ from actual situations.

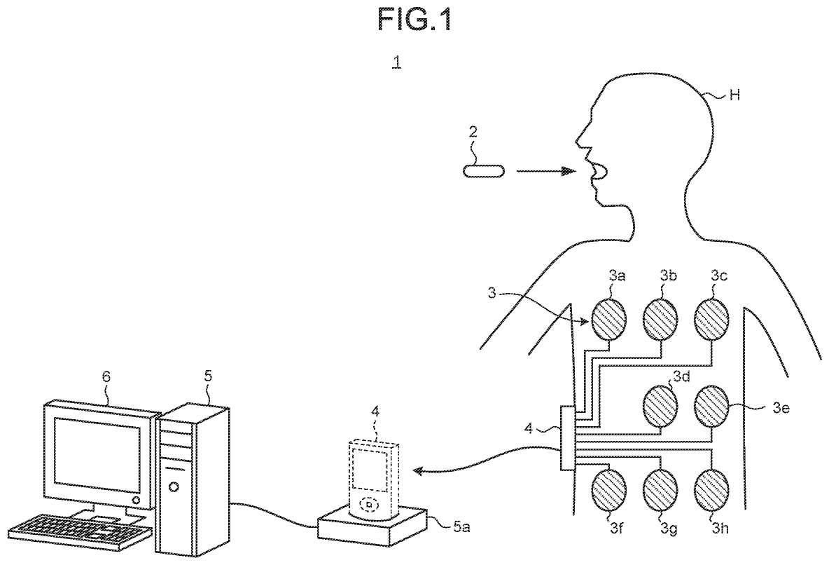

FIG. 1 is a schematic diagram illustrating a schematic configuration of a capsule endoscope system according to the embodiment. A capsule endoscope system 1 illustrated in FIG. 1 includes a capsule endoscope 2 that is introduced into a subject H and that generates image data by capturing an image inside the subject H, to transmit it by radio communication, a receiving device 4 that receives a radio signal transmitted from the capsule endoscope 2 through a receiving antenna unit 3 having plural receiving antennas 3a to 3h put on the subject H, and a processing device 5 that acquires an image signal captured by the capsule endoscope 2 from the receiving device 4 through a cradle 5a, and that processes the image signal to observe an image inside the subject H. The image processed by the processing device 5 is output, for example, from a display device 6 by displaying it.

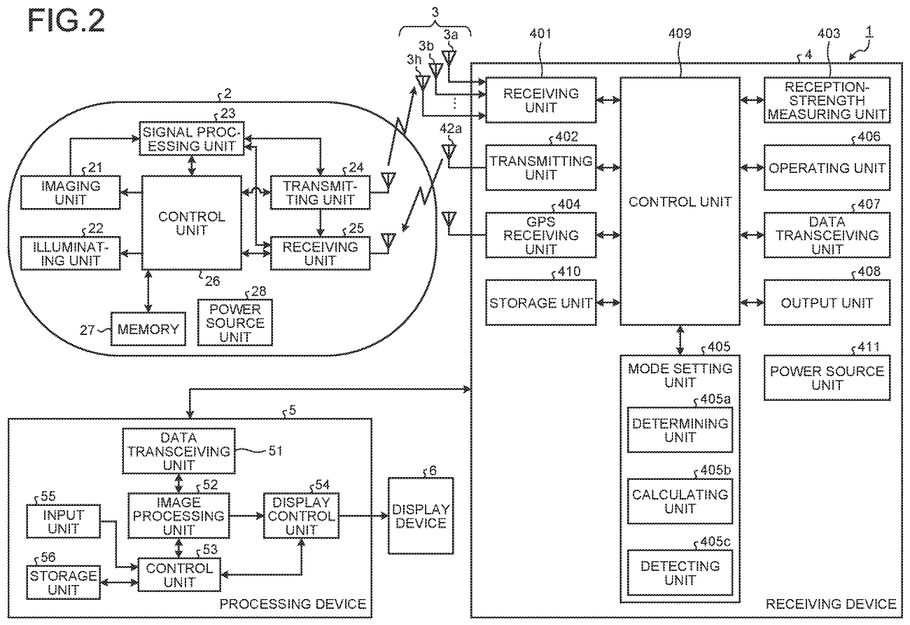

FIG. 2 is a block diagram illustrating a schematic configuration of the capsule endoscope system according to the embodiment. The capsule endoscope 2 includes an imaging unit 21, an illuminating unit 22, a signal processing unit 23, a transmitting unit 24, a receiving unit 25, a control unit 26, a memory 27, and a power source unit 28. The capsule endoscope 2 is a device including the respective components described above in a capsule-shaped casing in a size swallowable by the subject H.

The imaging unit 21 includes, for example, an imaging device that generates and outputs image data from imaging inside the subject H from an optical image formed on a light receiving surface, and an optical system, such as an object lens arranged on side of the light receiving surface of the imaging device. The imaging device has plural pixels receiving light from the subject arranged in a matrix form, and generates image data by performing photoelectric conversion with respect to the light received by the pixel. The imaging unit 21 reads a pixel value per horizontal line from the pixels arranged in a matrix form, and generates image data that includes plural pieces of line data to which a synchronization signal is added per horizontal line. The imaging unit 21 is constituted of a charge couple device (CCD), or a complementary metal oxide semiconductor (CMOS) imaging device.

The illuminating unit 22 is constituted of a white light emitting diode (LED) that emits white light being an illumination light, and the like. It may be configured to generate white light by coupling light of plural LEDs having different emitting wavelength bands or laser light sources, and the like, other than the white LED, or may be configured by using a xenon lamp, a halogen lamp, or the like.

The signal processing unit 23 performs predetermined signal processing with respect to the image data generated by the imaging unit 21, or to a control signal received by the receiving unit 25, by reading a predetermined program stored in the memory 27. For example, the signal processing unit 23 subjects image data acquired from the imaging unit 21 to A/D conversion processing, or to processing to convert into a predetermined format to transmit to the receiving device 4, or the like, and outputs the data to the transmitting unit 24. The signal processing unit 23 is implemented by a general purpose processor, such as a CPU, or a dedicated processor including various kinds of arithmetic circuits that perform specific functions, such as an ASIC.

The transmitting unit 24 subjects the image data output from the imaging unit 21 to modulation processing according to a set frequency, and transmits the data to the outside. The transmitting unit 24 acquires image data in a digital format, to superimpose related information, and transmits the data to the outside from a transmission antenna. The related information includes identification information (for example, serial number) of the capsule endoscope 2 assigned to identify an individual unit of the capsule endoscope 2, identification information (for example, captured image number) of image data to be transmitted, and the like. The transmitting unit 24 corresponds to a second transmitting unit.

The receiving unit 25 receives a control signal transmitted from the receiving device 4 through an antenna. The receiving unit 25 corresponds to a third receiving unit.

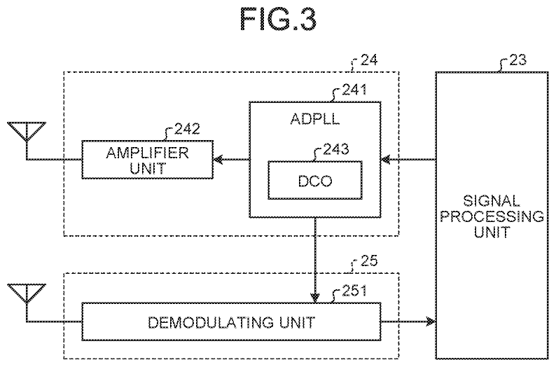

Configurations of the transmitting unit 24 and the receiving unit 25 will be explained, referring to FIG. 3. FIG. 3 is a block diagram illustrating a configuration of an essential part of the capsule endoscope in the capsule endoscope system according to the embodiment. The transmitting unit 24 includes an all-digital phase-locked loop (ADPLL) 241 and an amplifier unit 242.

The ADPLL 241 (completely digital phase synchronization circuit) is a phase synchronization circuit in which all components of the circuit are digitalized. The ADPLL 241 includes a digitally controlled oscillator (DCO) 243. The DCO 243 adjusts a frequency by a digital control based on a set frequency.

The ADPLL 241 modulates a signal while setting a frequency of a signal to be transmitted by the transmitting unit 24 to a frequency set by the receiving device 4, and outputs the modulated signal to the amplifier unit 242.

The amplifier unit 242 amplifies the signal input from the ADPLL 241 to a transmission power set in advance, to transmit to the outside.

The receiving unit 25 includes a demodulating unit 251. The demodulating unit 251 demodulates a signal (for example, a control signal) received from the receiving device 4. The demodulating unit 251 acquires information of set frequency from the ADPLL 241, and performs demodulation processing based on the acquired information.

The control unit 26 controls operation processing of the respective components of the capsule endoscope 2. The control unit 26 causes, for example, when the imaging unit 21 performs imaging processing, the imaging device to perform exposure processing and read-out processing, and causes the illuminating unit 22 to illuminate illumination light according to exposure timing of the imaging unit 21. The control unit 26 is constituted of a general purpose processor, such as CPU, or a dedicated processor including various kinds of arithmetic circuits that perform specific functions, such as ASIC.

The memory 27 stores an operation program for the control unit 26 to perform various kinds of operations, a control program, and parameters such as a threshold. The memory 27 is constituted of a volatile memory, a non-volatile memory, or a combination of those. Specifically, the memory 27 is constituted of a random access memory (RAM), a read only memory (ROM), and the like.

The power source unit 28 includes a battery constituted of a button battery or the like, a power source circuit that supplies power to respective parts, and a power switch that switches on and off states of the power source unit 28, and supplies power to the respective parts in the capsule endoscope 2 after the power switch is turned on. The power switch is constituted of, for example, a lead switch with which on and off states may be switched by an external magnetic force, and it may be switched to the on state by externally applying a magnetic force to the capsule endoscope 2 before using the capsule endoscope 2 (before the subject H swallows).

The capsule endoscope 2 as described sequentially captures images of a digestive tract(esophagus, stomach, small intestine, large intestine, and the like) at predetermined cycle (for example, 0.5 second cycle) while moving inside a digestive tract of the subject H by peristaltic movement of organs after it is swallowed by the subject H. Image signals acquired by this imaging operation and related information are sequentially transmitted to the receiving device 4 by radio communication. At this time, the capsule endoscope 2 switches a frequency of a signal to be transmitted according to frequency that is changed appropriately according to a place of the subject H.

The receiving device 4 includes a receiving unit 401, a transmitting unit 402, a reception-strength measuring unit 403, a GPS receiving unit 404, a mode setting unit 405, an operating unit 406, a data transceiving unit 407, an output unit 408, a control unit 409, a storage unit 410, and a power source unit 411. The receiving device 4 switches a frequency to be used for radio communication according to a communication state of GPS signal. Specifically, in the present embodiment, a first frequency set within a range of 305 MHz to 325 MHz, and a second frequency set within a range of 423 MHz to 443 MHz are switched. In the following, an example in which the first frequency is set to 315 MHz, and the second frequency is set to 433 MHz will be explained.

The receiving unit 401 receives a radio signal transmitted by the capsule endoscope 2 by radio communication. Specifically, image data and related information transmitted from the capsule endoscope 2 by radio communication are received through the receiving antenna unit 3. The receiving unit 401 performs predetermined signal processing, such as demodulation processing, with respect to the received image data. The receiving unit 401 corresponds to a first receiving unit.

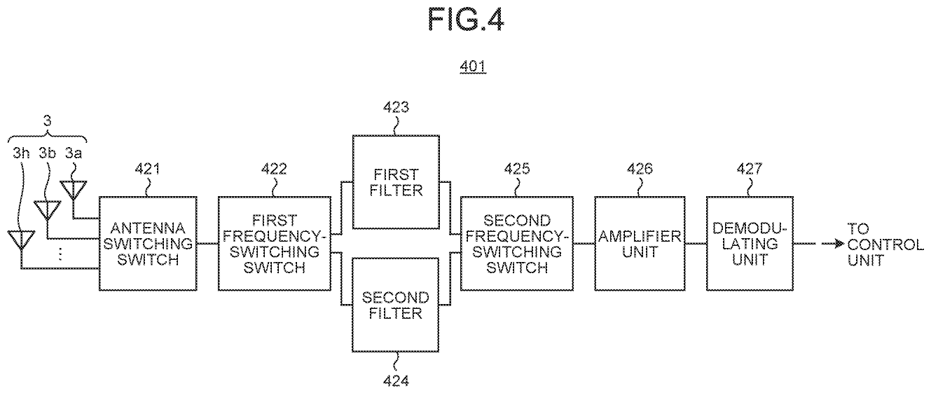

A configuration of the receiving unit 401 will be explained, referring to FIG. 4. FIG. 4 is a block diagram illustrating a configuration of an essential part of the receiving device in the capsule endoscope system according to the embodiment. The receiving unit 401 includes an antenna switching switch 421, a first frequency-switching switch 422, a first filter 423, a second filter 424, a second frequency-switching switch 425, an amplifier unit 426, and a demodulating unit 427. The receiving unit 401 is constituted of one or more units of a general purpose processor, such as CPU, and a dedicated processor including various kinds of arithmetic circuits that perform specific functions, such as ASIC.

The antenna switching switch 421 switches an antenna to receive a signal. Specifically, the antenna switching switch 421 causes respective receiving antennas to receive signals by sequentially switching a receiving antenna to be used for reception among receiving antennas 3a to 3h.

The first frequency-switching switch 422 switches, when a signal is input from the receiving antenna selected by the antenna switching switch 421, a transmission path to either one of a transmission path through the first filter 423, and a transmission path through the second filter 424 according to a set frequency. In the present embodiment, either frequency of 315 MHz and 433 MHz is used, and a transmission path is selected according to respective frequencies.

The first filter 423 is a bandpass filter that passes a signal of 315 MHz.

The second filter 424 is a bandpass filter that passes a signal of 433 MHz.

The second frequency-switching switch 425 switches a transmission path to either one of a transmission path entering through the first filter 423, and a transmission path entering through the second filter 424.

The amplifier unit 426 amplifies a signal that has passed through the second frequency-switching switch 425 to a gain set in advance, to input to the demodulating unit 427.

The demodulating unit 427 demodulates the signal (for example, a control signal) received from the receiving device 4. The demodulating unit 427 performs demodulation processing based on a frequency according to a mode set by the mode setting unit 405.

Returning back to FIG. 2, the transmitting unit 402 subjects information to be transmitted to a capsule, such as error information of an image output from the control unit 409 and mode change information of a capsule, to modulation processing, to transmit to the capsule endoscope 2 by an antenna 42a. The transmitting unit 402 corresponds to a first transmitting unit.

The reception-strength measuring unit 403 measures a received signal strength of a radio signal received by the receiving antennas 3a to 3h. The reception-strength measuring unit 403 is constituted of a general purpose processor, such as CPU, and a dedicated processor including various kinds of arithmetic circuits that perform specific functions, such as ASIC.

The GPS receiving unit 404 is constituted of a GPS receiver that receives a radio wave from a global positioning system (GPS) satellite. The GPS receiving unit 404 measures a position at a time of reception of the signal based on received positioning information, and outputs a result of positioning to the control unit 409 as position information. The positioning by the GPS receiving unit 404 may be performed by using a publicly-known method. The GPS receiving unit 404 corresponds to a second receiving unit. The GPS receiving unit 404 may receive positioning information from a positioning system, such as “Galileo” and “BeiDou System”, or may receive positioning information from a positioning system of a base station of a mobile telephone network.

The mode setting unit 405 switches a frequency to be used for communication with the capsule endoscope 2 by changing a mode based on a reception state of the GPS receiving unit 404, position information detected from the GPS receiving unit 404, and a state of a radio interference at a predetermined frequency. The mode setting unit 405 includes a determining unit 405a that determines magnitude relationship by comparing a value of a subject to be determined and a threshold level which is stored in the storage unit 410, a calculating unit 405b that calculates a parameter for radio interference detection, and a detecting unit 405c that detects radio interference based on the parameter for radio interference detection. The mode setting unit 405 is implemented by a general purpose processor, such as CPU, and a dedicated processor including various kinds of arithmetic circuits that perform specific functions, such as ASIC.

The operating unit 406 is an input device that is used to input various kinds of setting information and instruction information to the receiving device 4 by a user. The operating unit 406 is constituted of, for example, a switch, a button, and the like arranged on an operating panel of the receiving device 4.

The data transceiving unit 407 transmits image data and related information stored in the storage unit 410 to the processing device 5 when connected to the processing device 5 in a communication enabled state. The data transceiving unit 407 is constituted of a communication interface, such as LAN.

The output unit 408 is constituted of the means of display, beep, illuminate, and vibrations. The output unit 408 displays a notification according to an interference level, or output sound, light, and vibrations. The output unit 408 is constituted of at least one of a display, such as a liquid crystal display and an organic electroluminescence (EL) display, a speaker, a light emitting device, and a vibration generator, such as a vibration motor.

The control unit 409 controls the respective components of the receiving device 4. The control unit 409 is constituted of a general purpose processor, such as a CPU, or a dedicated processor including various kinds of arithmetic circuits that perform specific functions, such as an ASIC.

The storage unit 410 stores a program to operate the receiving device 4 to perform various functions, image data acquired by the capsule endoscope 2, a threshold for determination processing, map information (coordinate information according to latitude and longitude), an interference table to detect a radio interference, and the like. The storage unit 410 is constituted of a RAM, a ROM, or the like.

The power source unit 411 supplies power to the respective components of the receiving device 4. The power source unit 411 is implemented by a battery constituted of a cell.

The receiving device 4 as described above is put on the subject H and carried while capturing of the images of the digestive tract by the capsule endoscope 2. The receiving device 4 stores image data received through the receiving antenna unit 3 in the storage unit 410 in this period.

After the capsule endoscope is excreted, the receiving device 4 is taken out from the subject H, and is set to the cradle 5a (refer to FIG. 1) connected to the processing device 5. Thus, the receiving device 4 is connected to the processing device 5 in a communication enabled state, and transfers (downloads) the image data and the related information stored in the storage unit 410 to the processing device 5.

The processing device 5 is constituted of a work station having the display device 6, such as a liquid crystal display. The processing device 5 includes a data transceiving unit 51, an image processing unit 52, a control unit 53, a display control unit 54, an input unit 55, and a storage unit 56.

The data transceiving unit 51 is connected to the receiving device 4 through the cradle 5a, and performs transmission and reception of data with the receiving device 4. The data transceiving unit 51 is constituted of a communication interface, such as a universal serial bus (USB) and local area network (LAN).

The image processing unit 52 performs predetermined image processing to generate an image corresponding to image data input from the data transceiving unit 51 or image data stored in a storage unit 58, by executing a predetermined program stored in the storage unit 58. The image processing unit 52 is implemented by a general purpose processor, such as a CPU, or a dedicated processor including various kinds of arithmetic circuits that perform specific functions, such as an ASIC.

The control unit 53 performs instruction to the respective components constituting the processing device 5, transfer of data, and the like based on a signal input by an input unit 57 or image data input from the data transceiving unit 51, by executing various programs stored in the storage unit 56 to control overall operation of the processing device 5. The control unit 53 is implemented by a general purpose processor, such as a CPU, or a dedicated processor including various kinds of arithmetic circuits that perform specific functions, such as an ASIC.

The display control unit 54 subjects an image generated by the image processing unit 52 to predetermined processing, such as decimation of the image data according to a display size on the display device 6, tone adjustment, and the like, and then causes the display device 6 to display the thus acquired image together with information of an object to be displayed, such as a final score. The display control unit 54 is implemented, for example, by a general purpose processor, such as a CPU, or a dedicated processor including various kinds of arithmetic circuits that perform specific functions, such as an ASIC.

The input unit 55 accepts an input of information or a command according to an operation made by a user. The input unit 55 is implemented by, for example, an input device, such as a keyboard, a mouse, a touch panel, and various kinds of switches.

The storage unit 56 stores a program to operate the processing device 5 to perform various functions, various kinds of information used while the program is executed, image data and related information acquired from the receiving device 4, an endoscopic image that is generated by the image processing unit 52, and the like. The storage unit 56 is implemented by a semiconductor memory, such as a flash memory, a RAM, a ROM, a recording medium, such as an HDD, an MO, a CD-R, a DVD-R, and a driving device that drives the recording medium, and the like.

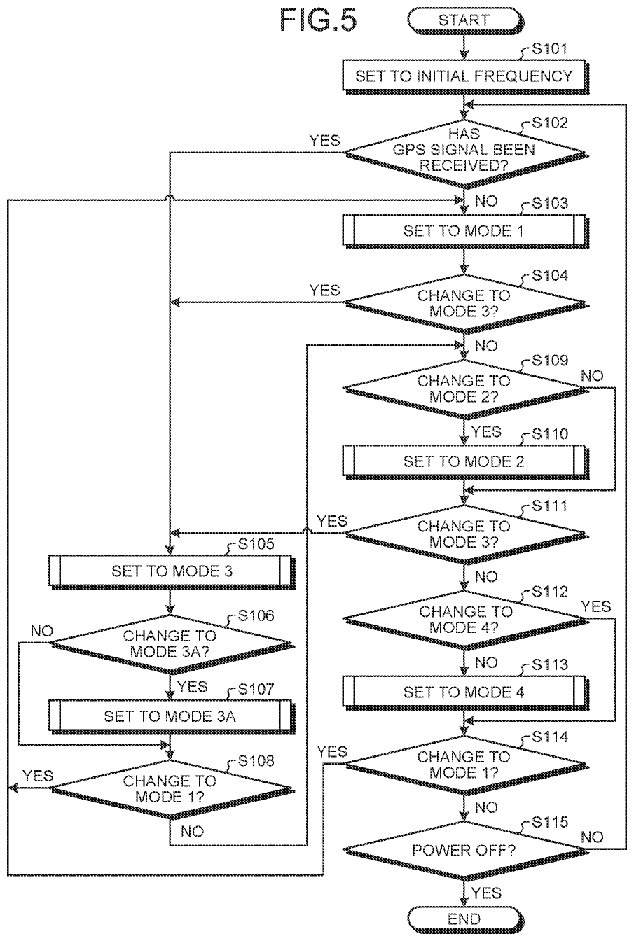

Subsequently, the mode setting processing relating to transmission and reception of image data performed by the capsule endoscope system 1 will be explained. FIG. 5 is a flowchart of the mode setting processing performed by the capsule endoscope system according to the embodiment.

First, at step S101, the receiving device 4 sets a frequency to be used for transmission and reception with the capsule endoscope 2 to an initial frequency. The initial frequency is, for example, 315 MHz.

At step S102 subsequent to step S101, the control unit 409 of the receiving device 4 determines whether the GPS receiving unit 404 has received a GPS signal (positioning information). When the control unit 409 determines that the GPS signal has not been received (step S102: NO), then transition to step S103. Moreover, when the control unit 409 determines that the GPS signal has been received (step S102: YES), then transition to step S105.

At step S103, the mode setting unit 405 sets the mode for performing transmission and reception of a signal to Mode 1. The processing of Mode 1 will be described later.

At step S104 subsequent to step S103, the control unit 409 determines whether processing of changing to Mode 3 is necessary by the processing of Mode 1. When the control unit 409 determines that setting change to Mode 3 is necessary (step S104: YES), then transition to step S105.

On the other hand, when the control unit 409 determines that the setting change to Mode 3 is not necessary (step S104: NO), then transition to step S109.

At step S105, the mode setting unit 405 sets the mode for performing transmission and reception of a signal to Mode 3. The processing of Mode 3 will be described later.

At step S106 subsequent to step S105, the control unit 409 determines whether processing of changing to Mode 3A is necessary by the processing of Mode 3. When the control unit 409 determines that the setting change to Mode 3A is necessary (step S106: YES), then transition to step S107.

On the other hand, when the control unit 409 determines that the change processing to Mode 3A is not necessary (step S106: NO), then transition to step S108.

At step S107, the mode setting unit 405 sets the mode for performing transmission and reception of a signal to Mode 3A. The processing of Mode 3A will be described later.

At step S108 subsequent to step S107, the control unit 409 determines whether processing of changing to Mode 1 is necessary. When the control unit 409 determines that the setting change to Mode 1 is necessary (step S108: YES), it returns to step S103.

On the other hand, when the control unit 409 determines that the setting change to Mode 1 is not necessary (step S108: NO), then transition to step S109.

At step S109, the control unit 409 determines whether processing of changing to Mode 2 is necessary. When the control unit 409 determines that the setting change to Mode 2 is necessary (step S109: YES), then transition to step S110.

On the other hand, when the control unit 409 determines that the setting change to Mode 2 is not necessary (step S109: NO), then transition to step S111.

At step S110, the mode setting unit 405 sets the mode for performing transmission and reception of a signal to Mode 2. The processing of Mode 2 will be described later.

At step S111, the control unit 409 determines whether processing of changing to Mode 3 is necessary. When the control unit 409 determines that the setting change to Mode 3 is necessary (step S111: YES), then transition to step S105.

On the other hand, when the control unit 409 determines that the setting change to Mode 3 is not necessary (step S111: NO), then transition to step S112.

At step S112, the control unit 409 determines whether processing of changing to Mode 4 is necessary. When the control unit 409 determines that the setting change to Mode 4 is necessary (step S112: YES), then transition to step S113.

On the other hand, when the control unit 409 determines that the setting change to Mode 4 is not necessary (step S112: NO), then transition to step S114.

AT step S113, the mode setting unit 405 sets the mode for performing transmission and reception of a signal to Mode 4. The processing of Mode 4 will be described later.

At step S114, when the control unit 409 determines whether processing of changing to Mode 1 is necessary. When the control unit 409 determines that the setting change to Mode 1 is necessary (step S114: YES), it returns to step S103.

On the other hand, when the control unit 409 determines that the setting change to Mode 1 is not necessary (step S114: NO), then transition to step S115.

At step S115, the control unit 409 determines whether to turn off the power source. Specifically, the control unit 409 determines whether a signal to turn off the power source has been input. When the control unit 409 determines that the signal to turn off the power source has been input (step S115: YES), the mode setting processing is ended.

On the other hand, when the control unit 409 determines that the signal to turn off the power source has not been input (step S115: NO), it returns to step S102.

In the present embodiment, a frequency to be used for radio communication is switched by monitoring a communication state of a signal at 315 MHz and/or 433 MHz, a reception state of positioning information from the positioning system (GPS), and by setting the communication mode (Modes 1 to 4 in this example) according to the communication state and the reception state. The communication state is determined by using a signal level of the frequency being used, and a detection result of a radio interference of a frequency not being used. Specifically, first, the mode for performing transmission and reception of a signal is set to Mode 1 or Mode 3 based on presence or absence of a GPS signal. Thereafter, the mode setting unit 405 changes the mode according to a change mode determined based on the communication state and the reception state in the processing in the respective modes. At this time, the frequency setting information is transmitted to the capsule endoscope 2 from the receiving device 4, and the frequency to be used for communication is switched also in the capsule endoscope 2.

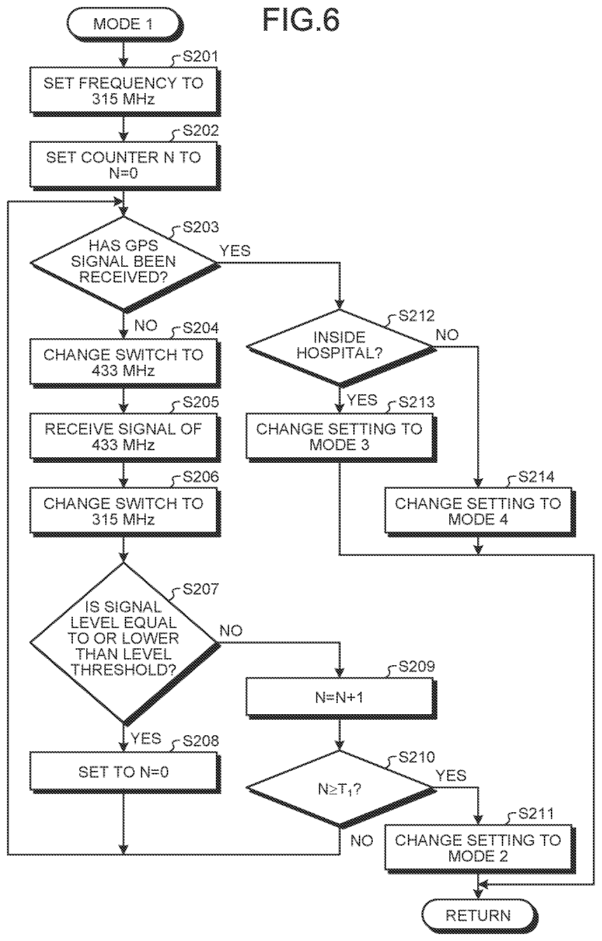

Subsequently, the processing of Modes 1 to 4 will be explained, referring to FIG. 6 to FIG. 10. FIG. 6 is a flowchart of processing of Mode 1 in the mode setting processing performed by the capsule endoscope system according to the embodiment.

In Mode 1, first, the mode setting unit 405 sets the frequency to be used for transmission and reception of a signal to 315 MHz (step S201). At this time, when the frequency has already been set to 315 MHz, the mode setting unit 405 maintains the setting. Moreover, in Mode 1, the transmission path in which the signal passes through the first filter 423 (signal of 315 MHz passes) is set by the first frequency-switching switch 422 and the second frequency-switching switch 425.

At step S202 subsequent to step S201, the mode setting unit 405 sets a counter N relating to a signal level to N=0.

At step S203 subsequent to step S202, the mode setting unit 405 determines whether the GPS receiving unit 404 has received a GPS signal. Specifically, the mode setting unit 405 determines whether the GPS receiving unit 404 has received a GPS signal through the control unit 409. When the mode setting unit 405 determines that a GPS signal has not been received (step S203: NO), then transition to step S204. Moreover, when the mode setting unit 405 determines that a GPS signal has been received (step S203: YES), then transition to step S212.

At step S204, the mode setting unit 405 switches the switch of the receiving unit 401. Specifically, the mode setting unit 405 switches the first frequency-switching switch 422 and the second frequency-switching switch 425 under control of the control unit 409, and changes to the transmission path in which a signal passes through the second filter 424 (signal of 433 MHz passes).

After the switch is switched, the receiving unit 401 receives a signal of 433 MHz (step S205). The mode setting unit 405 acquires a signal received by the receiving unit 401.

At step S206 subsequent to step S205, the mode setting unit 405 switches the switch of the receiving unit 401. Specifically, the mode setting unit 405 sets back to the transmission path in which a signal passes through the first filter 423 (signal of 315 MHz passes) by switching the first frequency-switching switch 422 and the second frequency-switching switch 425 under control of the control unit 409.

At step S207, the mode setting unit 405 determines whether the signal level of the signal of 433 MHz is equal to or lower than a threshold. Specifically, the determining unit 405a determines whether a strength of a signal acquired at step S205 and measured by the reception-strength measuring unit 403 (hereinafter, this is referred to as signal level) is equal to or lower than a level threshold set in advance. The level threshold is set to, for example, a minimum strength out of signal strengths determined that an interference has not occurred at 433 MHz.

When the mode setting unit 405 determines that the signal level of the signal of 433 MHz is equal to or lower than the threshold (step S207: YES), then transition to step S208. On the other hand, when the mode setting unit 405 determines that the signal level of the signal of 433 MHz is higher than the threshold (step S207: NO), then transition to step S209.

At step S208, the mode setting unit 405 sets the counter N to N=0. The control unit 409 returns to step S203 after the counter setting.

At step S209, the mode setting unit 405 increments the counter N by 1.

At step S210 subsequent to step S209, the mode setting unit 405 determines whether it may be determined that an interference is absent in a signal based on the counter N after the increment. Specifically, the determining unit 405a determines whether the counter N after the increment is equal to or larger than a threshold T₁ for interference check. This threshold T₁ is set to, for example, a confirmation number of times that enables to determine that an interference has not occurred in a signal at 433 MHz.

When the determining unit 405a determines that the counter N is not equal to or larger than the threshold T₁ (step S209: NO), the mode setting unit 405 returns to step S203. On the other hand, when the determining unit 405a determines that the counter N is equal to or larger than the threshold T₁ (step S209: YES),the mode setting unit 405 shifts to step S211.

At step S211, the mode setting unit 405 determines to change the setting mode to Mode 2, and returns to the flowchart in FIG. 5.

On the other hand, at step S212, the mode setting unit 405 determines whether the receiving device 4 (the subject H) is positioned in a hospital based on the acquired GPS signal. Specifically, the detecting unit 405c detects a position (coordinates) of the receiving device 4 from the position information acquired from the GPS signal, and determines whether the detected position is inside the hospital from the map information stored in the storage unit 410.

When the mode setting unit 405 determines that the receiving device 4 (the subject H) is positioned in the hospital (step S212: YES), then transition to step S213. On the other hand, when the mode setting unit 405 determines that the receiving device 4 (the subject H) is not positioned in the hospital (step S212: NO), then transition to step S214.

At step S213, the mode setting unit 405 determines to change the setting mode to Mode 3, and returns to the flowchart in FIG. 5.

At step S214, the mode setting unit 405 determines to change the setting mode to Mode 4, and returns to the flowchart in FIG. 5.

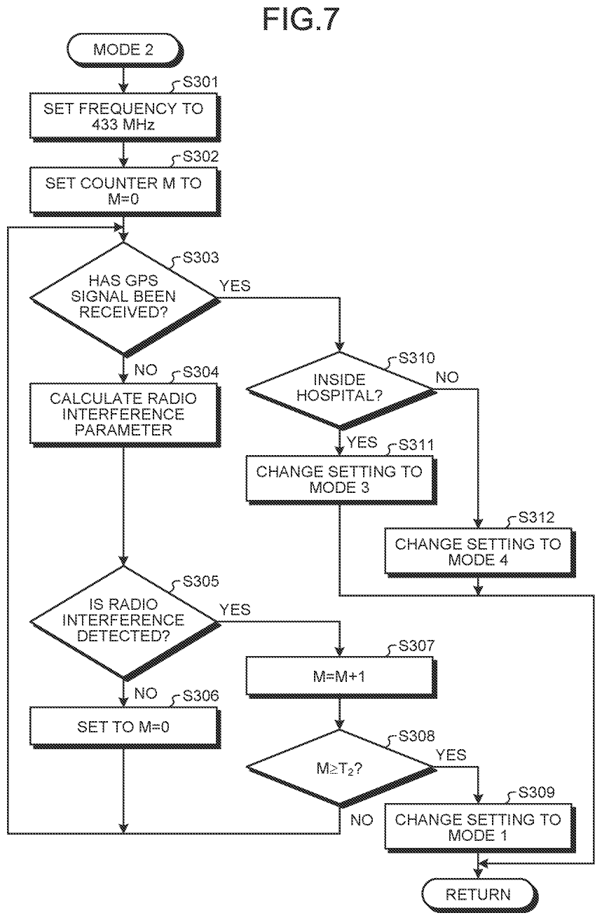

Subsequently, the processing of Mode 2 will be explained, referring to FIG. 7. FIG. 7 is a flowchart of Mode 2 in the mode setting processing performed by the capsule endoscope system according to the embodiment.

In Mode 2, first, the mode setting unit 405 sets the frequency to be used for transmission and reception of a signal to 433 MHz (step S301). At this time when the frequency has already been set to 433 MHz, the mode setting unit 405 maintains the setting. Moreover, in Mode 2, the transmission path in which a signal passes through the second filter 424 (signal of 433 MHz passes) is set by the first frequency-switching switch 422 and the second frequency-switching switch 425.

At step S302 subsequent to step S301, the mode setting unit 405 sets a counter M relating to radio interference to M=0.

At step S303 subsequent to step S302, the mode setting unit 405 determines whether the GPS receiving unit 404 has received a GPS signal similarly to step S202 described above. When the mode setting unit 405 determines that a GPS signal has not been received (step S303: NO), then transition to step S304. Moreover, when the mode setting unit 405 determines that a GPS signal has been received (step S303: YES), then transition to step S310.

At step S304, the calculating unit 405b calculates a radio interference parameter to detect a radio interference. The radio interference parameter calculated herein includes a signal value of image data, a counter value of the synchronization signal, and the like.

At step S305 subsequent to step S304, the mode setting unit 405 performs detection of a radio interference. Specifically, the detecting unit 405c determines presence or absence of a radio interference based on the calculated radio interference parameter and an interference table that is set in advance, and stored in the storage unit 410. When a radio interference is not detected by the detecting unit 405c (step S305: NO), the mode setting unit 405 shifts to step S306. On the other hand, when a radio interference is detected by the detecting unit 405c (step S305: YES), the mode setting unit 405 shifts to step S307.

At step S306, the mode setting unit 405 sets the counter M to M=0. The control unit 409 returns to step S303 after the counter setting.

At step S307, the mode setting unit 405 increments the counter M by 1.

At step S308 subsequent to step S307, the mode setting unit 405 determines whether it may be determined that an interference is absent in a signal based on the counter M after the increment. Specifically, the determining unit 405a determines whether the counter M after the increment is equal to or larger than a threshold T₂ for interference check. This threshold T₂ is set to, for example, a confirmation number of times that enables to determine that an interference not recommended for use at 433 MHz has occurred. The threshold T₂ may be set to the same value as the threshold T₁ described above.

When the mode setting unit 405 determines that the counter M is smaller than the threshold T₂ (step S308: NO), it returns to step S303. On the other hand, when the mode setting unit 405 determines that the counter M is equal to or larger than the threshold T₂ (step S308: YES), then transition to step S309.

At step S309, the mode setting unit 405 determines to change the setting mode to Mode 1, and returns to the flowchart in FIG. 5.

On the other hand, at step S310, the mode setting unit 405 determines whether the receiving device 4 (the subject H) is positioned in a hospital based on the acquired GPS signal, similarly to step S212 described above.

When the mode setting unit 405 determines that the receiving device 4 (the subject H) is positioned in the hospital (step S310: YES), then transition to step S311. On the other hand, when the mode setting unit 405 determines that the receiving device 4 (the subject H) is not positioned in the hospital (step S310: NO), then transition to step S312.

At step S311, the mode setting unit 405 determines to change the setting mode to Mode 3, and returns to the flowchart in FIG. 5.

At step S312, the mode setting unit 405 determines to change the setting mode to Mode 4, and returns to the flowchart in FIG. 5.

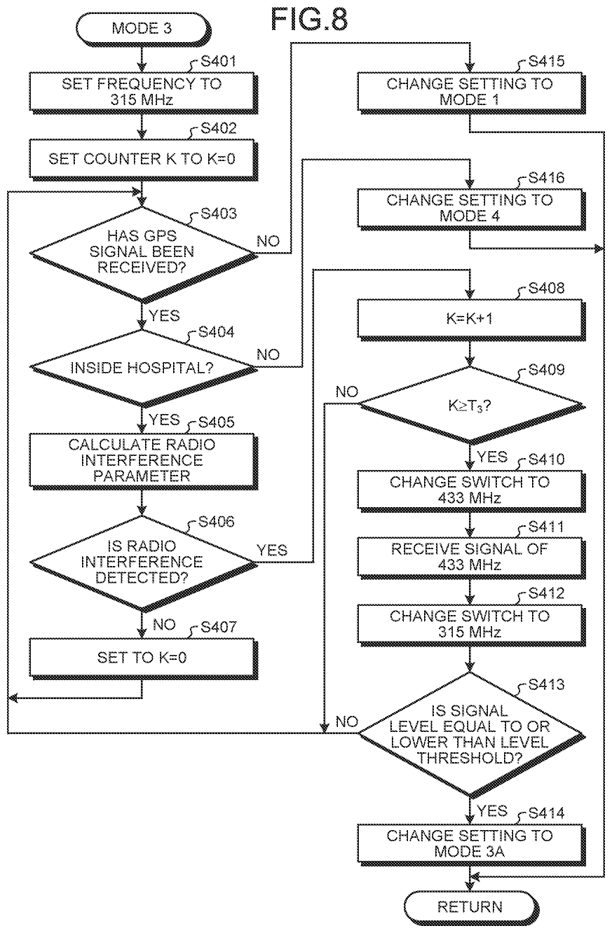

Subsequently, the processing of Mode 3 will be explained, referring to FIG. 8. FIG. 8 is a flowchart of Mode 3 in the mode setting processing performed by the capsule endoscope system according to the embodiment.

In Mode 3, first, the mode setting unit 405 sets the frequency to be used for transmission and reception of a signal to 315 MHz (step S401). At this time when the frequency has already been set to 315 MHz, the mode setting unit 405 maintains the setting. Moreover, in Mode 3, the transmission path in which a signal passes through the first filter 423 (signal of 315 MHz passes) is set by the first frequency-switching switch 422 and the second frequency-switching switch 425.

At step S402 subsequent to step S401, the mode setting unit 405 sets a counter K relating to radio interference to zero.

At step S403 subsequent to step S402, the mode setting unit 405 determines whether the GPS receiving unit 404 has received a GPS signal similarly to step S202 described above. When the mode setting unit 405 determines that a GPS signal has not been received (step S403: NO), then transition to step S415. Moreover, when the mode setting unit 405 determines that a GPS signal has been received (step S403: YES), then transition to step S404.

At step S404, the mode setting unit 405 determines whether the receiving device 4 (the subject H) is positioned in a hospital based on the acquired GPS signal, similarly to step S212 described above.

When the mode setting unit 405 determines that the receiving device 4 (the subject H) is positioned in the hospital (step S404: YES), then transition to step S405. On the other hand, when the mode setting unit 405 determines that the receiving device 4 (the subject H) is not positioned in the hospital (step S404: NO), then transition to step S416.

At step S405, the calculating unit 405b calculates a radio interference parameter to detect a radio interference.

At step S406 subsequent to step S405, the mode setting unit 405 performs detection of a radio interference similarly to step S305 described above. When a radio interference is not detected (step S406: NO), the mode setting unit 405 shifts to step S407. On the other hand, a radio interference is detected (step S406: YES), the mode setting unit 405 shifts to step S408.

At step S407, the mode setting unit 405 sets the counter K to zero. The control unit 409 returns to step S403 after the counter setting.

At step S408, the mode setting unit 405 increments the counter K by 1.

At step S409 subsequent to step S408, the mode setting unit 405 determines whether it may be determined that an interference is absent in a signal based on the counter K after the increment. Specifically, the determining unit 405a determines whether the counter K after the increment is equal to or larger than a threshold T₃ for interference check. This threshold T₃ is set to, for example, a confirmation number of times that enables to determine that an interference not recommended for use at 315 MHz has occurred.

When the mode setting unit 405 determines that the counter K is smaller than the threshold T₃ (step S409: NO), it returns to step S403. On the other hand, when the mode setting unit 405 determines that the counter K is equal to or larger than the threshold T₃ (step S409: YES), then transition to step S410.

At step S410, the mode setting unit 405 switches the switch of the receiving unit 401. Specifically, the mode setting unit 405 changes to the transmission path in which a signal passes through the second filter 424 (signal of 433 MHz passes) by switching the first frequency-switching switch 422 and the second frequency-switching switch 425.

After the switching of the switch, the receiving unit 401 receives a signal of 433 MHz (step S411). The mode setting unit 405 acquires the signal received by the receiving unit 401.

At step S412 subsequent to step S411, the mode setting unit 405 switches the switch of the receiving unit 401. Specifically the mode setting unit 405 returns to the transmission path in which a signal passes through the first filter 423 (signal of 315 MHz passes) by switching the first frequency-switching switch 422 and the second frequency-switching switch 425.

At step S413, the mode setting unit 405 determines whether the signal level of the signal of 433 MHz is equal to or lower than a threshold. Specifically, the determining unit 405a determines whether a level of a signal that is acquired at step S411 and that is measured by the reception-strength measuring unit 403 is equal to or lower than a level threshold that is set in advance. The level threshold is set to, for example, a minimum strength out of signal strengths determined that an interference has not occurred at 433 MHz, similarly to step S207.

When the determining unit 405a determines that the signal level of the signal of 433 MHz is equal to or lower than the threshold (step S413: NO), the mode setting unit 405 shifts to step S414. On the other hand, when the determining unit 405a determines that the signal level of the signal of 433 MHz is higher than the threshold (step S413: YES), the mode setting unit 405 returns to step S403.

At step S414, the mode setting unit 405 determines to change the setting mode to Mode 3A, and returns to the flowchart in FIG. 5.

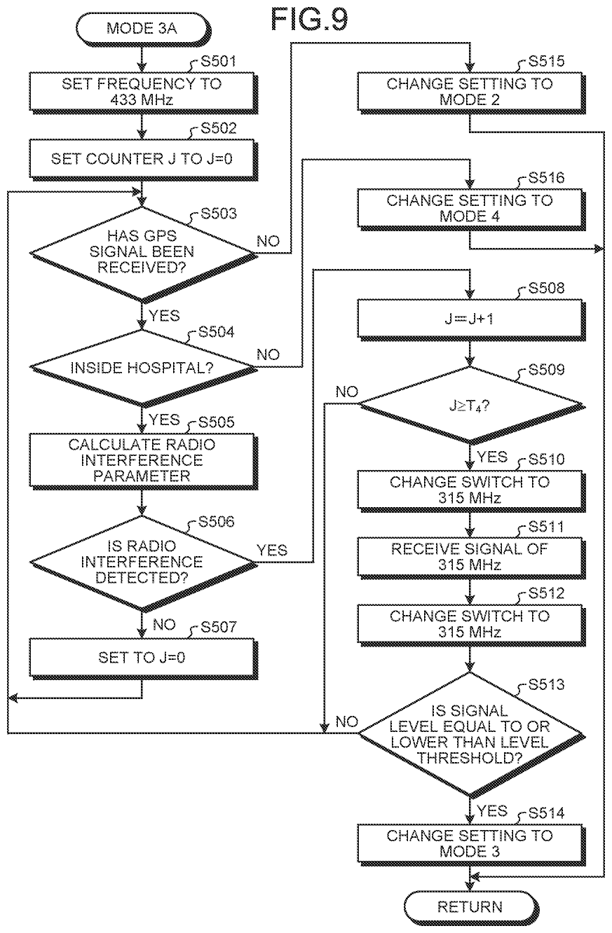

Subsequently, the processing of Mode 3A will be explained, referring to FIG. 9. FIG. 9 is a flowchart of mode 3A in the mode setting processing performed by the capsule endoscope system according to the embodiment.

In Mode 3A, first, the mode setting unit 405 sets the frequency to be used for transmission and reception of a signal to 433 MHz (step S501). Moreover, in Mode 3A, the transmission path in which a signal passes through the second filter 424 (signal of 433 MHz passes) is set by the first frequency-switching switch 422 and the second frequency-switching switch 425.

At step S502 subsequent to step S501, the mode setting unit 405 sets a counter J relating to a radio interference to zero.

At step S503 subsequent to step S502, the mode setting unit 405 determines whether the GPS receiving unit 404 has received a GPS signal, similarly to step S202 described above. When it is determined that a GPS signal has not been received (step S503: NO), the mode setting unit 405 shifts to step S515. Moreover when it is determined that a GPS signal has been received (step S503: YES), the mode setting unit 405 shifts to step S504.

At step S504, the mode setting unit 405 determines whether the receiving device 4 (the subject H) is positioned in a hospital based on the acquired GPS signal similarly to step S212 described above.

When the mode setting unit 405 determines that the receiving device 4 (the subject H) is positioned in the hospital (step S504: YES), then transition to step S505. On the other hand, when the mode setting unit 405 determines that the receiving device 4 (the subject H) is not positioned in the hospital (step S504: NO), then transition to step S516.

At step S505, the calculating unit 405b calculates a radio interference parameter to detect a radio interference.

At step S506 subsequent to step S505, the mode setting unit 405 performs detection of a radio interference, similarly to step S305 described above. When a radio interference is not detected (step S506: NO), the mode setting unit 405 shifts to step S507. On the other hand, when a radio interference is detected (step S506: YES), the mode setting unit 405 shifts to step S508.

At step S507, the mode setting unit 405 sets a counter J to zero. The control unit 409 returns to step S504 after the counter setting.

At step S508, the mode setting unit 405 increments the counter J by 1.

At step S509 subsequent to step S508, the mode setting unit 405 determines whether it may be determined that an interference is absent in a signal based on the counter J after the increment. Specifically, the determining unit 405a determines whether the counter J after the increment is equal to or larger than a threshold T₄ for interference check. This threshold T₄ is set to, for example, a confirmation number of times that enables to determine that an interference not recommended for use at 433 MHz has occurred. The threshold T₄ may be set to the same value as the threshold T₁ and the threshold T₂ described above.

When the mode setting unit 405 determines that the counter J is smaller than the threshold T₄ for interference check (step S509: NO), it returns to step S503. On the other hand, when the mode setting unit 405 determines that the counter j is equal to or larger than the threshold T₄ (step S509: YES), then transition to step S510.

At step S510, the mode setting unit 405 switches the switch of the receiving unit 401. Specifically, the mode setting unit 405 changes to the transmission path in which a signal passes through the first filter 423 (signal of 315 MHz passes) by switching the first frequency-switching switch 422 and the second frequency-switching switch 425 under control of the control unit 409.

After the switching of the switch, the receiving unit 401 receives a signal of 315 MHz (step S511). The mode setting unit 405 acquires the signal received by the receiving unit 401.

At step S512 subsequent to step S511, the mode setting unit 405 switches the switch of the receiving unit 401. Specifically, the mode setting unit 405 returns to the transmission path in which a signal passes through the second filter 424 (signal of 315 MHz passes) by switching the first frequency-switching switch 422 and the second frequency-switching switch 425 under control of the control unit 409.

At step S513, the mode setting unit 405 determines whether a signal level of the signal of 315 MHz is equal to or lower than a threshold. Specifically, the determining unit 405a determines whether the signal level acquired at step S511, and measured by the reception-strength measuring unit 403 is equal to or lower than the level threshold set in advance. The level threshold is set to, for example, a minimum strength out of signal strengths determined that an interference has not occurred at 315 MHz.

When the determining unit 405a determines that the signal level of the signal of 433 MHz is equal to or lower than the threshold (step S513: YES), the mode setting unit transition to step S514. On the other hand, when the determining unit 405a determines that the signal level of the signal of 433 MHz is larger than the threshold (step S513: NO), the mode setting unit 405 returns to step S503.

At step S514, the mode setting unit 405 determines to change the setting mode to Mode 3, and returns to the flowchart in FIG. 5.

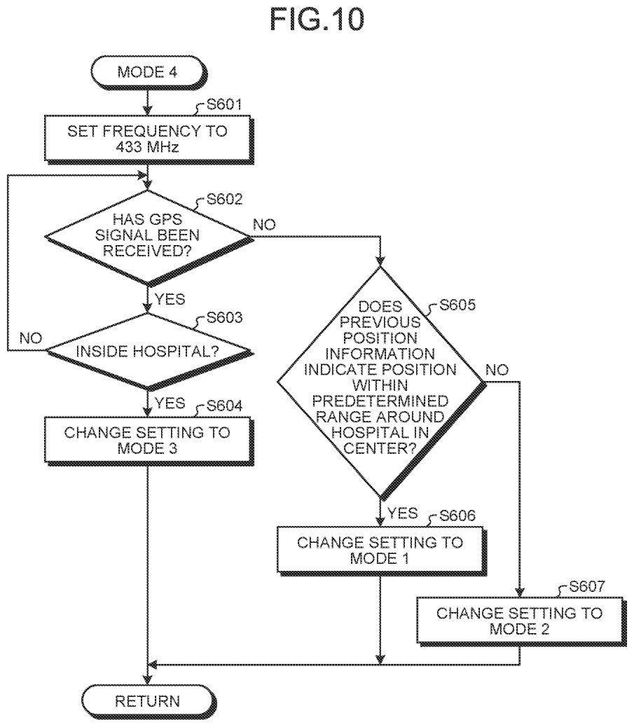

Subsequently, the processing of Mode 4 will be explained, referring to FIG. 10. FIG. 10 is a flowchart of mode 4 in the mode setting processing performed by the capsule endoscope system according to the embodiment.

In Mode 4, first, the mode setting unit 405 sets the frequency to be used for transmission and reception of a signal to 433 MHz (step S601). At this time, when the frequency has already been set to 433 MHz, the mode setting unit 405 maintains the setting. Moreover, in Mode 4, the transmission path in which a signal passes through the second filter 424 (signal of 433 MHz passes) is set by the first frequency-switching switch 422 and the second frequency-switching switch 425.

At step S602 subsequent to step S601, the mode setting unit 405 determines whether the GPS receiving unit 404 has received a GPS signal, similarly to step S202 described above. When it is determined that a GPS signal has been received (step S602: YES), the mode setting unit 405 shifts to step S603. Moreover, when it is determined that a GPS signal has not been received (step S602: NO), the mode setting unit 405 shifts to step S605.

At step S603, the mode setting unit 405 determines whether the receiving device 4 (the subject H) is positioned in a hospital based on the acquired GPS signal similarly to step S212 described above.

When The mode setting unit 405 determines that the receiving device 4 (the subject H) is positioned in the hospital (step S603: YES), then transition to step S604. On the other hand, when the mode setting unit 405 determines that the receiving device 4 (the subject H) is not positioned in the hospital (step S603: NO), then transition to step S602.

At step S604, the mode setting unit 405 determines to change the setting mode to Mode 3, and returns to the flowchart in FIG. 5.

Moreover, at step S605, the mode setting unit 405 determines whether previous position information indicates a position inside a predetermined range around the hospital in center. Specifically, the detecting unit 405c detects whether the position of the receiving unit 401 is a position in the predetermined range from the hospital based on latest position information and the map information. The predetermined range is, for example, range within R meters (m) radius of the center of premises of the hospital. R is set to, for example, 50 m.

When the detecting unit 405c detects that the position of the receiving unit 401 is within the predetermined range from the hospital (step S605: YES), the mode setting unit 405 shifts to step S606. On the other hand, when the detecting unit 405c detects that the position of the receiving unit 401 is not within the predetermined range from the hospital (step S605: NO), the mode setting unit 405 shifts to step S607.

At step S606, the mode setting unit 405 determines to change the setting mode to Mode 1, and returns to the flowchart in FIG. 5.

On the other hand, at step S607, the mode setting unit 405 determines to change the setting mode to Mode 2, and returns to the flowchart in FIG. 5.

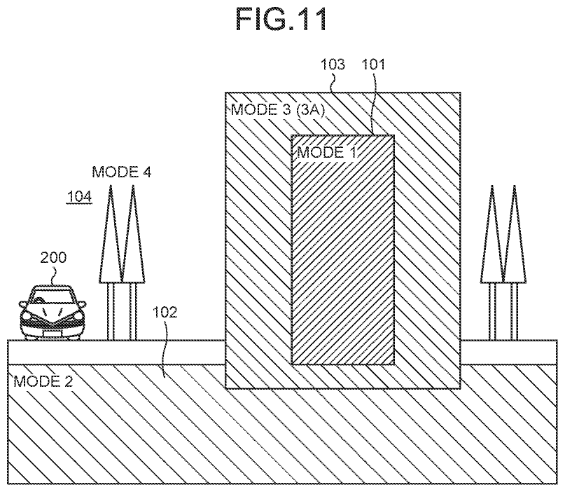

By the mode change processing explained above, the frequency to be used for communication with the capsule endoscope 2 is appropriately changed according to a position of the receiving device 4 (the subject H). That is, the frequency to be used for transmission and reception is switched according to an external environment in which the subject H is present. FIG. 11 is a diagram for explaining a mode that may be set in an external environment. In FIG. 11, a region 101 is an area deep inside in a hospital, and is a region in which a device that operates at a frequency of 400 MHz band, such as a medical telemeter, is used. Moreover, a region 102 is a basement of the hospital, and is a region in which a GPS signal cannot be received. A region 103 is a region in which a device that operates at a frequency of 400 MHz band described above is not used. Furthermore, a region 104 is a region in which a car 200 having a keyless entry system and a tire monitoring system that operate at a frequency of 300 MHz runs.

In the region 101, Mode 1 is mainly set. This is because a device that operates at a frequency of 400 MHz band is present therein, and communications are performed at 315 MHz to avoid interference with the device.

In the region 102, Mode 2 is mainly set. This is because a GPS signal is not received therein, assuming that the subject H is located in a place in which a GPS signal cannot reach, such as basement, and communications are performed at 433 MHz to avoid interference with, for example, the car 200 parked in a basement parking lot.

In the region 103, Mode 3 (or 3A) is mainly set. This is because it is assumed that a device that operates at a frequency of 400 MHz is be not present, and a device that operates at a frequency of 300 MHz is not present either, and communications are performed at 315 MHz or 433 MHz depending on a degree of interference of radio waves.

In the region 104, Mode 4 is mainly set. This is because a device (for example, the car 200) that operates at a frequency of 300 MHz is present therein, and communications are performed at 433 MHz to avoid interference with the device.

In the above embodiment, it is configured to appropriately switch frequencies to be used for communication, or modes among Modes 1 to 4 in which flows to check an interference in communication differ according to a position of the receiving device 4 (the subject H). According to the present embodiment, a radio interference in communications between the capsule endoscope 2 and the receiving device 4 may be suppressed.

The embodiment to implement the present disclosure has been explained so far, but the present disclosure is not to be limited only to the embodiment and modifications described above. The present disclosure is not limited to the embodiment and the modifications described above, but may include various embodiments within a scope not departing a technical ideas described in claims. Moreover, the components of the embodiment and the modification may be combined appropriately.

Furthermore, in the present embodiment, it has been explained that an antenna for reception is separately arranged, and has an independent transmitting unit and receiving unit, but it is not limited thereto. For example, it may be configured to perform transmission and reception with a single antenna.

Moreover, operating programs for respective processing performed by the respective components of the capsule endoscope 2, the receiving device 4, and the processing device 5 of the capsule endoscopes system 1 according to the present embodiment may be configured to be provided in a computer-readable recording medium, such as a CD-ROM, a flexible disk (FD), a CD-R, and a DVD, stored therein in an installable format or an executable format file, or may be configured to be stored in a computer connected to a network, such as the Internet, and to be distributed by being downloaded through the network. Moreover, it may be configured to be provided or distributed through a network, such as the Internet.

The capsule endoscope system and the receiving device are useful for suppressing a radio interference in communications between an image acquiring device and a receiving device.

According to the present disclosure, an effect of suppressing a radio interference in communications between an image acquiring device and a receiving device is produced.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general concept as defined by the appended claims and their equivalents.

What is claimed is:

1. A receiving device for transmitting and receiving a radio signal by radio communication with a capsule endoscope introduced inside a subject, the receiving device comprising:
a first receiver configured to selectively receive the radio signal by switching between a first frequency and a second frequency that is different from the first frequency;
a second receiver configured to receive positioning information from an external positioning system;
a processor comprising hardware, the processor being configured to
acquire information indicating whether the positioning information has been received from the positioning system, and a signal level of a signal at, at least one of the first frequency and the second frequency, and
set to either one of a mode in which the first frequency is used for the radio communication and a mode in which the second frequency is used for the radio communication; and
a first transmitter configured to transmit information of frequency according to the set mode to the capsule endoscope.

2. The receiving device according to claim 1, wherein the processor is configured to:
monitor a signal level of a radio signal at the second frequency in a state set to the mode in which the first frequency is used; and
set to the mode in which the second frequency is used when the second receiver has not received the positioning information, and number of times of monitoring in which the signal level of the second frequency is equal to or lower than a threshold is equal to or more than number of times set in advance.

3. The receiving device according to claim 1, wherein the processor is configured to:
monitor a radio interference in a radio signal of the second frequency in a state set to the mode in which the second frequency is used; and
set to the mode in which the first frequency is used when the second receiver has not received the positioning information, and number of detection of the radio interference is equal to or more than number set in advance.

4. The receiving device according to claim 1, wherein the processor is configured to:
monitor a radio interference in a radio signal of the first frequency, and a signal level of a radio signal of the second frequency in a state set to the mode in which the first frequency is used; and
set to the mode in which the second frequency is used when the second receiver has received the positioning information, and number of detection of the radio interference is equal to or more than number set in advance, and number of times of monitoring in which the signal level of the second frequency is equal to or lower than a threshold is equal to or more than number of times set in advance.

5. The receiving device according to claim 1, wherein the processor is configured to:
monitor a radio interference in a radio signal of the second frequency, and a signal level of a radio signal of the first frequency in a state set to the mode in which the second frequency is used; and
set to the mode in which the first frequency is used when the second receiver has received the positioning information, and number of detection of the radio interference is equal to or more than number set in advance, and number of monitoring in which the signal level of the first frequency is equal to or lower than a threshold is equal to or more than number of times set in advance.

6. The receiving device according to claim 4, wherein the processor is configured to:
determine whether the receiving device is positioned in a hospital based on position information of the hospital in which introduction of the capsule endoscope into the subject is started, and on position information of the receiving device based on the positioning information; and
monitor a radio interference in a radio signal of the first frequency and a signal level of a radio signal of the second frequency when it is determined that the receiving device is positioned in the hospital in a state set to the mode in which the first frequency is used.

7. The receiving device according to claim 4, wherein the processor is configured to:
determine whether the receiving device is positioned in a hospital based on position information of the hospital in which introduction of the capsule endoscope into the subject is started, and on position information of the receiving device based on the positioning information; and
monitor a radio interference in a radio signal of the second frequency and a signal level of a radio signal of the first frequency when it is determined that the receiving device is positioned in the hospital in a state set to the mode in which the second frequency is used.

8. The receiving device according to claim 1, wherein
the first frequency is set within a range of 305 MHz to 325 MHz, and
the second frequency is set within a range of 423 MHz to 443 MHz.

9. A capsule endoscope system comprising:
a capsule endoscope adapted to be introduced into a subject; and
a receiving device configured to transmit and receive a radio signal by radio communication with a capsule endoscope introduced inside a subject, the receiving device comprising:
a first receiver configured to selectively receive the radio signal by switching between a first frequency and a second frequency that is different from the first frequency;
a second receiver configured to receive positioning information from an external positioning system;
a processor comprising hardware, the processor being configured to
acquire information indicating whether the positioning information has been received from the positioning system, and a signal level of a signal at, at least one of the first frequency and the second frequency, and
set to either one of a mode in which the first frequency is used for the radio communication and a mode in which the second frequency is used for the radio communication; and
a first transmitter configured to transmit information of frequency according to the set mode to the capsule endoscope,
wherein the capsule endoscope comprises:
a third receiver configured to receive information of the frequency from the first transmitter; and
a second transmitter configured to
select either one of the first frequency and the second frequency according to the information of the frequency received by the third receiver, and
transmit the signal at the selected frequency.

10. A control method executed by a receiving device configured to transmit and receive a radio signal by radio communication with a capsule endoscope introduced inside a subject, the method comprising:
selectively receiving the radio signal while switching between a first frequency and a second frequency that is different from the first frequency;
receiving positioning information from an external positioning system;
acquiring information indicating whether the positioning information has been received from the positioning system, and a signal level of a signal at, at least one of the first frequency and the second frequency, to set to either one of a mode in which the first frequency is used for the radio communication and a mode in which the second frequency is used for the radio communication; and
transmitting information of a frequency according to the set mode to the capsule endoscope.
